(19) United States
(12) Patent Application Publication
(10) Pub. No.: US 2007/0277329 A1
(43) Pub. Date: Dec. 6, 2007

(54) Dyeing Composition Comprising at Least One Dye Precursor and One Amphiphilic Block Copolymer

(76) Inventors: Claude Dubief, Le Chesnay (FR); Frederic Guerin, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/557,984

(22) PCT Filed: Apr. 30, 2004
(86) PCT No.: PCT/EP04/06068

§ 371 Date: Oct. 25, 2006

Related U.S. Application Data
(60) Provisional application No. 60/513,594, filed on Oct. 24, 2003.

(30) Foreign Application Priority Data
May 23, 2003 (FR) 0306248

(51) Int. Cl. A61K 8/00

(52) U.S. Cl. 8/405; 524/588

(57) ABSTRACT

A subject-matter of the present invention is a dyeing composition comprising, in an appropriate medium, at least one dye precursor, and at least one water-soluble or water-dispersible amphiphilic block copolymer comprising at least one hydrophilic block and at least one hydrophobic block without a fatty chain of more than 8 carbon atoms, the hydrophilic block or blocks representing at least 30% by weight of the block copolymer, with the exception of block copolymers of ethylene oxide and of propylene oxide, block copolymers comprising urethane units and block copolymers comprising siloxane units. The composition of the present invention makes it possible in particular to obtain an intense colouring of the keratinous fibres while retaining a soft feel and ready disentangling subsequent to the colouring.

A subject-matter of the invention is a dyeing composition comprising at least one dye precursor and one amphiphilic block copolymer.

Another subject-matter of the invention is the use of this composition for the dyeing of keratinous fibres and the dyeing process employing this composition.

It is known to dye keratinous fibres and in particular human hair with dyeing compositions comprising oxidation dye precursors, generally known as oxidation bases, such as ortho- or para-phenylenediamines, ortho- or para-aminophenols and heterocyclic compounds. These oxidation bases are colourless or weakly coloured compounds which, in combination with oxidizing substances, can give rise by an oxidative coupling process to coloured compounds.

It is also known that the hues obtained with these oxidation bases can be varied by combining them with couplers or colouring modifiers, the latter being chosen in particular from aromatic meta-diamines, meta-aminophenols, meta-diphenols and certain heterocyclic compounds, such as indole compounds.

The variety of the molecules employed as oxidation bases and couplers makes it possible to obtain a rich palette of colours.

The “permanent” colouring obtained by virtue of these oxidation dyes has, however, to satisfy a certain number of requirements. Thus, it must be without disadvantage toxicologically, it must make it possible to obtain hues with the desired intensity and it must behave well in the face of external agents, such as light, bad weather, washing, permanent waving, perspiration and rubbing.

The dyes must also make it possible to cover white hair and, finally, be as unselective as possible, that is to say make it possible to obtain the least possible differences in colouring along the same keratinous fibre, which is generally sensitized (i.e. damaged) to a varying degree between its tip and its root.

The various dyes or dye precursors are generally present in a dyeing medium which conventionally comprises water and optionally a solvent which makes possible better dissolution of the dyes or dye precursors. The medium also conventionally comprises agents, such as anionic, cationic or nonionic, amphoteric or zwitterionic surface-active agents or their mixtures, anionic, cationic, nonionic, amphoteric or zwitterionic polymers or their blends, inorganic or organic thickening agents and in particular anionic, cationic, nonionic and amphoteric associative polymer thickeners. These various constituents are added to the dyeing medium for various reasons, for example to thicken the dyeing medium, to protect, repair or condition the hair before, during or after the colouring, and the like.

The aim of the present invention is to provide novel compositions for dyeing the hair which make it possible to improve the intensity of the colour while retaining a soft feel and ready disentangling after colouring.

This aim is achieved with the present invention, a subject-matter of which is a dyeing composition comprising, in an appropriate medium,
at least one dye precursor, and
at least one water-soluble or water-dispersible amphiphilic block copolymer comprising at least one hydrophilic block and at least one hydrophobic block without a fatty chain of more than 8 carbon atoms, the hydrophilic block or blocks representing at least 30% by weight of the block copolymer, with the exception of block copolymers of ethylene oxide and of propylene oxide, block copolymers comprising urethane units and block copolymers comprising siloxane units.

Another subject-matter of the invention is the use of the composition of the present invention for dyeing keratinous fibres, in particular human keratinous fibres, such as the hair.

Another subject-matter of the invention is a dyeing process employing this composition.

The composition of the present invention makes it possible in particular to obtain an intense colouring of keratinous fibres while retaining a soft feel and ready disentangling subsequent to the colouring.

The block copolymers which can be used for the present invention are linear or star di-, tri- or multiblock copolymers. According to a preferred embodiment, the block copolymer of use in the composition of the invention is linear.

According to a specific embodiment, the block copolymers comprise at least two blocks with a hydrophilic nature, the two blocks having different chemical natures, one of them contributing water-solubility or water-dispersability to the block copolymer.

The block copolymers used in the present invention can be
diblock copolymers of formula AB, triblock copolymers of formula ABA or BAB, and
multiblock copolymers comprising at least two hydrophilic blocks and at least two hydrophobic blocks arranged alternately,
   each A representing a hydrophilic block and each B representing a hydrophobic block, it being possible for the A blocks of the same polymer to be identical or different and it being possible for the B blocks of the same polymer to be identical or different.

The diblock copolymers and the triblock copolymers comprising a hydrophilic central block and two hydrophobic side blocks are preferred in the context of the present invention.

The term “water-soluble or water-dispersible block copolymer” is understood to mean, within the meaning of the present invention, a copolymer which, at the concentration of 0.1% of active material in water at 25° C., results, spontaneously or after neutralization with an acid or a base, in a macroscopically homogeneous, transparent or translucent, solution or suspension, that is to say a solution or suspension having a value for the transmission at a wavelength of 500 nm through a sample with a thickness of 1 cm of at least 70%, preferably of approximately 80%.

The term “hydrophobic block” is understood to mean, according to the present invention, a block comprising at least 75 mol % of water-insoluble monomers. In other words, the block can comprise at most 25 mol % of one or more water-soluble monomers according to the definition given above. This proportion is preferably at most equal to 10 mol %. According to a particularly preferred embodiment, this proportion is less than or equal to 5 mol %.

The term “hydrophilic block” is understood to mean a block comprising at least 75 mol % of water-soluble monomers according to the definition given above with reference to the copolymer. In other words, the hydrophilic block can comprise at most 25 mol % of one or more water-insoluble monomers according to the definition given above. This proportion is preferably at most equal to 10 mol %. According to a particularly preferred embodiment, this proportion is less than or equal to 5 mol %.

According to a particularly preferred embodiment, the hydrophilic block or blocks represent at least 60% by weight of the block copolymer.

The water-soluble monomers forming the hydrophilic block of the block copolymers used in the present invention can be chosen from anionic water-soluble monomers, nonionic water-soluble monomers and cationic water-soluble monomers, or a mixture of these.

Mention may be made, as examples of anionic water-soluble monomers, of carboxylic acids comprising ethylenic unsaturation, such as acrylic acid, methacrylic acid, itaconic acid, fumaric acid, crotonic acid and maleic acid anhydride; 2-acrylamido-2-methylpropanesulphonic acid; styrenesulphonic acid; vinylsulphonic acid and vinylphosphonic acid.

The nonionic water-soluble monomers encompass, inter alia, acrylamide, N—(C₁₋₆ alkylated)- or N,N-di(C₁₋₃ alkylated)acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol (copolymerized in the form of vinyl acetate and then hydrolysed), ethylene oxide, hydroxyethyl acrylate, hydroxypropyl acrylate, hydroxyethyl methacrylate and hydroxypropyl methacrylate.

The cationic water-soluble monomers can be chosen from dimethyldiallylammonium chloride, methylvinylimidazolium chloride, 2-vinylpyridine, 4-vinylpyridine, 2-methyl-5-vinylpyridine, vinylamine, monomers of formula
H₂C═CR₁—CO—X₂
in which
R₁ represents a hydrogen atom or a methyl group,
X₂ represents a linear or branched C₁₋₆ hydrocarbonaceous group carrying at least one primary, secondary or tertiary amine functional group or at least one quaternary nitrogen atom, or a group of formula NHR₂ or of formula NR₂R₃ where R₂ and R₃ each represent, independently of one another, a linear or branched C₁₋₆ hydrocarbonaceous group carrying at least one primary, secondary or tertiary amine functional group or at least one quaternary nitrogen atom.

The water-insoluble monomers forming the hydrophobic block of the block copolymers are preferably chosen from vinylaromatic monomers, such as styrene and its alkylated derivatives, for example 4-butylstyrene, α-methylstyrene and vinyltoluene, dienes, such as butadiene and 1,3-hexadiene, and alkylated derivatives of dienes, such as isoprene and dimethylbutadiene, chloroprene, C₁₋₁₀ alkyl, C₆₋₁₀ aryl or C₆₋₁₀ aralkyl acrylates and C₁₋₁₀ alkyl, C₆₋₁₀ aryl or C₆₋₁₀ aralkyl methacrylates, such as, for example, methyl, ethyl, n-butyl, 2-ethylhexyl, tert-butyl, isobornyl, phenyl or benzyl(meth)acrylates, vinyl acetate, vinyl ethers of formula CH₂═CH—O—R and allyl ethers of formula CH₂═CH—CH₂—O—R where R represents a C₁₋₆ alkyl group, acrylonitrile, vinyl chloride, vinylidene chloride, caprolactone, ethylene, propylene, or fluorovinyl monomers or vinyl monomers with a perfluorinated chain, such as fluoroalkyl acrylates and methacrylates or alkyl α-fluoroacrylates.

The block copolymers used in the present invention also, of course, encompass those in which the hydrophilic block and the hydrophobic block are composed exclusively of water-soluble monomers and water-insoluble monomers respectively. These blocks can be homopolymer blocks or copolymer blocks including two or more than two different monomers of the same type.

The number-average molecular mass of each block, whether hydrophobic or hydrophilic, copolymer or homopolymer, is preferably between 500 and 100 000, in particular between 500 and 50 000, with a polydispersity index (M_{w}/Mₙ) of between 1.01 and 3.0, preferably between 1.1 and 2.5.

The ratio by weights of the hydrophobic blocks and the hydrophilic blocks of the block copolymer is preferably between 1/20 and 20/1 and in particular between 1/10 and 10/1.

The dyeing compositions of the present invention preferably comprise the diblock copolymers in the dissolved or finely dispersed state in the dyeing medium.

The block polymers of the invention can be prepared by the synthetic processes conventionally used to produce block polymers. Mention may be made, for example, of anionic or cationic polymerizations and controlled radical polymerization (see “New Method of Polymer Synthesis”, Blackie Academic & Professional, London, 1995, volume 2, page 1. or Trends Polym. Sci., 4, page 183 (1996) by C. J. Hawker), which controlled radical polymerization can be carried out according to different processes, such as, for example, atom transfer radical polymerization (ATRP) (see JACS, 117, page 5614 (1995), by Matyjasezwski et al.), or the method of radicals, such as nitroxides (Georges et al., Macromolecules, 1993, 26, 2987).

Use may also be made of these processes to produce just one of the two types of blocks of the polymer of the invention, the other block being introduced into the final polymer via the initiator used or else by coupling reaction between the hydrophilic and hydrophobic blocks.

The amount of amphiphilic block copolymers in the dyeing compositions of the present invention depends on a large number of parameters, among which may be mentioned the molecular mass of the copolymers, the number and size of the hydrophilic and hydrophobic blocks, the amount of polymers which are beneficial for the hair, and the viscosity of the composition which it is desired to obtain.

Generally, the block polymer or polymers are present in the composition of the present invention in an amount of between 0.01 and 20% by weight of active material, preferably between 0.1 and 5% by weight of active material, with respect to the weight of the dyeing composition.

The dye precursors of use in the present invention can be oxidation bases or couplers conventionally used in oxidation dyeing. According to a specific embodiment, the composition of the present invention comprises at least one oxidation base and at least one coupler.

By way of example, the oxidation bases can be chosen from para-phenylenediamines, bisphenylalkylenediamines, para-aminophenols, bis(para-aminophenol)s, ortho-aminophenols, heterocyclic bases and their addition salts.

Mention may be made, among para-phenylene-diamines, by way of example, of para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-(β-hydroxyethyl)-para-phenylene-diamine, 2-fluoro-para-phenylenediamine, 2-isopropyl-para-phenylenediamine, N-(β-hydroxypropyl)-para-phenylenediamine, 2-hydroxymethyl-para-phenylene-diamine, N,N-dimethyl-3-methyl-para-phenylenediamine, N,N-(ethyl, β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-(β-hydroxyethyloxy)-para-phenylenediamine, 2-(β-acetylaminoethyloxy)-para-phenylene-diamine, N-(β-methoxyethyl)-para-phenylenediamine, 4-aminophenylpyrrolidine, 2-thienyl-para-phenylenediamine, 2-(β-hydroxyethylamino)-5-aminotoluene, 3-hydroxy-1-(4′-aminophenyl)pyrrolidine, and their addition salts with an acid.

Among the para-phenylenediamines mentioned above, para-phenylenediamine, para-toluenediamine, 2-isopropyl-para-phenylenediamine, 2-(β-hydroxyethyl)-para-phenylenediamine, 2-(β-hydroxyethyloxy)-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-(β-acetylaminoethyloxy)-para-phenylenediamine, and their addition salts with an acid, are particularly preferred.

Mention may be made, among bisphenylalkylene-diamines, by way of example, of N,N′-bis(β-hydroxy-ethyl)-N,N′-bis(4′-aminophenyl)-1,3-diaminopropanol, N,N′-bis(β-hydroxyethyl)-N,N′-bis(4′-aminophenyl)-ethylenediamine, N,N′-bis(4-aminophenyl)tetra-methylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and their addition salts with an acid.

Mention may be made, among para-aminophenols, by way of example, of para-aminophenol, 4-amino-3-methylphenol, 4-amino-3-fluorophenol, 4-amino-3-(hydroxymethyl)phenol, 4-amino-2-methylphenol, 4-amino-2-(hydroxymethyl)phenol, 4-amino-2-(methoxymethyl)-phenol, 4-amino-(2-aminomethyl)phenol, 4-amino-2-[(p-hydroxyethyl)aminomethyl]phenol, 4-amino-2-fluorophenol, 4-amino-2-chlorophenol, 4-amino-2,6-dichlorophenol, 4-amino-6-[(5′-amino-2′-hydroxy-3′-methylphenyl)methyl]-2-methylphenol and bis(5-amino-2-hydroxyphenyl)methane, and their addition salts with an acid.

Mention may be made, among ortho-aminophenols, by way of example, of 2-aminophenol, 2-amino-5-methylphenol, 2-amino-6-methylphenol, 5-acetamido-2-aminophenol, and their addition salts with an acid.

Mention may be made, among heterocyclic bases, by way of example, of pyridine derivatives, pyrimidine derivatives and pyrazole derivatives.

Mention may be made, among pyridine derivatives, of the compounds disclosed, for example, in Patents GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid.

Other pyridine oxidation bases of use in the present invention are the oxidation bases 3-aminopyrazolo[1,5-a]pyridines or their addition salts disclosed, for example, in Patent Application FR 2 801 308. Mention may be made, by way of example, of pyrazolo[1,5-a]pyridin-3-ylamine; 2-(acetylamino)pyrazolo[1,5-a]pyridin-3-ylamine; 2-(morpholin-4-yl)pyrazolo[1,5-a]pyridin-3-ylamine; 3-aminopyrazolo[1,5-a]pyridine-2-carboxylic acid; 2-methoxypyrazolo[1,5-a]pyridin-3-ylamine; (3-aminopyrazolo[1,5-a]pyridin-7-yl)methanol; 2-(3-aminopyrazolo[1,5-a]pyridin-5-yl)ethanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]-pyridine-3,7-diamine; 7-(morpholin-4-yl)-pyrazolo[1,5-a]pyridin-3-ylamine; pyrazolo[1,5-a]-pyridine-3,5-diamine; 5-(morpholin-4-yl)-pyrazolo[1,5-a]pyridin-3-ylamine; 2-(3-aminopyrazolo[1,5-a]pyridin-5-yl)(2-hydroxyethyl)amino]ethanol; 2-[(3-aminopyrazolo[1,5-a]-pyridin-7-yl)(2-hydroxyethyl)amino]ethanol; 3-aminopyrazolo[1,5-a]pyridin-5-ol; 3-aminopyrazolo[1,5-a]pyridin-4-ol; 3-aminopyrazolo[1,5-a]pyridin-6-ol; 3-aminopyrazolo[1,5-a]pyridin-7-ol; and their addition salts with an acid or with a base.

Mention may be made, among pyrimidine derivatives, of the compounds disclosed, for example, in Patents DE 2 359 399, JP 88-169 571, JP 05-63 124 or EP 0 770 375 or Patent Application WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine or 2,5,6-triamino-pyrimidine, and pyrazolopyrimidine derivatives, such as those mentioned in Patent Application FR-A-2 750 048, among which may be mentioned pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo-[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)-amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]-pyrimidine-3,7-diamine, 3-amino-5-methyl-7-(imidazolyl-propylamino)pyrazolo[1,5-a]pyrimidine, and their addition salts with an acid and their tautomeric forms, when a tautomeric equilibrium exists.

Mention may be made, among pyrazole derivatives, of the compounds disclosed in Patents DE 3 843 892 and DE 4 133 957 and Patent Applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diamino-pyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methyl-pyrazole, 4,5-diamino-1-(β-hydroxyethyl)-3-methyl-pyrazole, 4,5-diamino-1-ethyl-3-methylpyrazole, 4,5-diamino-1-ethyl-3-(4′-methoxyphenyl)pyrazole, 4,5-diamino-1-ethyl-3-(hydroxymethyl)pyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)-amino-1,3-dimethylpyrazole, 3,4,5-triaminopyrazole, 1-methyl-3,4,5-triaminopyrazole, 3,5-diamino-1-methyl-4-(methylamino)pyrazole, 3,5-diamino-4-(β-hydroxyethyl)-amino-1-methylpyrazole, and their addition salts with an acid.

The amount of each of the oxidation bases is generally between 0.001 and 10% by weight approximately of the total weight of the dyeing composition, preferably between 0.005 and 5%.

Mention may in particular be made, among the couplers which can be used in the composition of the present invention, of meta-phenylenediamines, meta-aminophenols, meta-diphenols, naphthalene couplers, heterocyclic couplers, and their addition salts.

Mention may be made, by way of example, of 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(5-hydroxyethylamino)-1-methoxybenzene, 1,3-diamino-benzene, 1,3-bis(2,4-diaminophenoxy)propane, 3-ureidoaniline, 3-ureido-1-(dimethylamino)benzene, sesamol, α-naphthol, 2-methyl-1-naphthol, 6-hydroxy-indole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis-(β-hydroxyethylamino)toluene, and their addition salts with an acid.

In the composition of the present invention, the amount of each of the couplers is generally between 0.001 and 10% by weight approximately of the total weight of the dyeing composition, preferably between 0.005 and 5%.

Generally, the addition salts of the oxidation bases and of the couplers which can be used in the context of the invention are chosen in particular from the addition salts with an acid, such as hydrochlorides, hydrobromides, sulphates, citrates, succinates, tartrates, lactates, tosylates, benzene-sulphonates, phosphates and acetates, and the addition salts with a base, such as sodium hydroxide, potassium hydroxide, ammonia, amines or alkanolamines.

The dyeing composition according to the invention can additionally comprise one or more direct dyes which can be chosen in particular from dyes conventionally used in direct dyeing and among which may be mentioned all the aromatic and/or nonaromatic dyes commonly used, such as nitro and amine dyes, phenols, aminophenols, naphthols, methines, azomethines, nitros, aromatic diamines, nitrosos, styryls, triarylmethanes, diarylmethanes, azos, anthraquinone and naphthoquinone dyes, porphyrins, tetraphenylporphyrins, metalloporphyrins, phthalocyanines, methinecyanines, natural dyes of carotenoid, terpenoid or flavonoid type, fluorescent molecules (fluorescein, rhodamine, coumarin, and the like), and others.

These direct dyes can be of nonionic, anionic or cationic nature.

In addition to the block copolymer, the dyeing medium, also referred to as dyeing vehicle, generally comprises water or a mixture of water and of at least one organic solvent, in order to dissolve the compounds which would not be sufficiently soluble in water. Mention may be made, as organic solvent, for example, of lower C₁-C₄ alkanols, such as ethanol and isopropanol; polyols and polyol ethers, such as 2-butoxyethanol, propylene glycol, propylene glycol monomethyl ether, diethylene glycol monoethyl ether and diethylene glycol monomethyl ether; and aromatic alcohols, such as benzyl alcohol or phenoxyethanol; and their mixtures.

The solvents are preferably present in proportions preferably of between 1 and 40% by weight approximately with respect to the total weight of the dyeing composition and more preferably still between 5 and 30% by weight approximately.

The dyeing composition in accordance with the invention can also include various adjuvants conventionally used in hair dyeing compositions, such as anionic, cationic, nonionic, amphoteric or zwitterionic surface-active agents or their mixtures, anionic, cationic, nonionic, amphoteric or zwitterionic polymers or their blends, other than the block polymers of use in the present invention, inorganic or organic thickening agents and in particular anionic, cationic, nonionic and amphoteric associative polymer thickeners, antioxidants, penetration agents, sequestering agents, fragrances, buffers, dispersing agents, conditioning agents, such as, for example, volatile or nonvolatile and modified or unmodified silicones, film-forming agents, ceramides, preservatives or opacifying agents.

The above adjuvants are generally present in an amount of, for each of them, between 0.01 and 20% by weight with respect to the weight of the composition.

Of course, a person skilled in the art will take care to choose this or these optional additional compounds so that the advantageous properties intrinsically attached to the oxidation dyeing composition in accordance with the invention are not, or not substantially, detrimentally affected by the envisaged addition or additions.

The pH of the dyeing composition in accordance with the invention is generally between 3 and 12 approximately and preferably between 5 and 11 approximately. It can be adjusted to the desired value by means of acidifying or basifying agents commonly used in dyeing keratinous fibres or alternatively using conventional buffer systems.

Mention may be made, among acidifying agents, by way of example, of inorganic or organic acids, such as hydrochloric acid, orthophosphoric acid, sulphuric acid, carboxylic acids, such as acetic acid, tartaric acid, citric acid or lactic acid, or sulphonic acids.

Mention may be made, among basifying agents, by way of example, of aqueous ammonia, alkaline carbonates, alkanolamines, such as mono-, di- and triethanolamines, and their derivatives, sodium hydroxide, potassium hydroxide and the compounds of following formula (II): 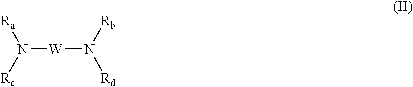
in which W is a propylene residue optionally substituted by a hydroxyl group or a C₁-C₄ alkyl radical and Rₐ, R_{b}, R_{c} and R_{d}, which are identical or different, represent a hydrogen atom or a C₁-C₄ alkyl or C₁-C₄ hydroxyalkyl radical.

The dyeing composition according to the invention can be provided in various forms, such as in the form of liquids, creams or gels or in any other form appropriate for carrying out dyeing of keratinous fibres and in particular of human hair.

The process of the present invention is a process in which the composition according to the present invention as defined above is applied to the fibres in the presence of an oxidizing agent for a time sufficient to develop the desired colouring. The colour can be developed at acidic, neutral or alkaline pH and the oxidizing agent can be added to the composition of the invention only at the time of use or it can be employed from an oxidizing composition comprising it, applied simultaneously with or sequentially to the composition of the invention.

According to a specific embodiment, the composition according to the present invention is mixed, preferably at the time of use, with a composition comprising, in a medium appropriate for dyeing, at least one oxidizing agent, this oxidizing agent being present in an amount sufficient to develop a colouring. The mixture obtained is subsequently applied to the keratinous fibres. After a leave-in time of 3 to 50 minutes approximately, preferably 5 to 30 minutes approximately, the keratinous fibres are rinsed, washed with a shampoo, rinsed again and then dried.

The oxidizing agents conventionally used for the oxidation dyeing of keratinous fibres are, for example, hydrogen peroxide, urea hydrogen peroxide, alkali metal bromates, persalts, such as perborates and persulphates, peracids and oxidase enzymes, among which may be mentioned peroxidases, 2-electron oxidoreductases, such as uricases, and 4-electron oxygenases, such as laccases. Hydrogen peroxide is particularly preferred.

The oxidizing composition can also include various adjuvants conventionally used in hair dyeing compositions and as defined above.

The pH of the oxidizing composition including the oxidizing agent is such that, after mixing with the dyeing composition, the pH of the resulting composition applied to the keratinous fibres preferably varies between 3 and 12 approximately, and more preferably still between 5 and 11. It can be adjusted to the desired value by means of acidifying or basifying agents commonly used in dyeing keratinous fibres and as defined above.

According to another embodiment, the process of the invention comprises the application to the keratinous fibres of a composition comprising the dye precursor, in the presence of an oxidizing agent or of an oxidizing composition, for a time sufficient to develop the desired colouring, this application being followed by the application of the block copolymer.

According to one alternative form, the block copolymer is present in the oxidizing composition.

The ready-to-use composition which is finally applied to the keratinous fibres can be provided in various forms, such as in the form of liquids, of creams or of gels or in any other form appropriate for carrying out dyeing of keratinous fibres and in particular of human hair.

Another subject-matter of the invention is a dyeing multi-compartment device or “kit” in which a first compartment includes the dyeing composition of the present invention defined above and a second compartment includes an oxidizing agent.

According to a specific embodiment, the kit of the invention comprises a first compartment which comprises a dyeing composition comprising the dye precursor, a second compartment comprising the oxidizing agent and a third compartment which comprises the block copolymer. This device can be equipped with a means allowing the desired mixture to be delivered to the hair, such as the devices disclosed in Patent FR-2 586 913 on behalf of the Applicant Company.

The examples which follow serve to illustrate the invention without, however, exhibiting a limiting nature.

### EXAMPLES

### Dyeing Examples

The following colouring compositions were prepared:

| | Ex. 1 | Ex. 2 | Ex. 3 | Ex. 4 | Ex. 5 |
|---|---|---|---|---|---|
| Hexylene glycol | 12.5 g | | | | |
| Propylene glycol | 12.5 g | | | | |
| Ethanol | 10 g | | | | |
| Sodium | 0.30 g | | | | |
| metabisulphite | | | | | |
| para- | 0.49 g | | | | |
| Phenylenediamine | | | | | |
| 1-Methyl-2-hydroxy- | 0.55 g | | | | |
| 4-aminobenzene | | | | | |
| Polymer 1 (comp.) | 1 g | | | | |
| Polymer 2 | | 1 g | | | |
| Polymer 3 | | | 1 g | | |
| Polymer 4 | | | | 1 g | |
| Polymer 5 | | | | | 1 g |
| Polymer 6 | | | | | |
| Aqueous ammonia | 10 g | 10 g | 10 g | 10 g | 10 g |
| Water | q.s. | q.s. | q.s. | q.s. | q.s. |
| | for | for | for | for | for |
| | 100 g | 100 g | 100 g | 100 g | 100 g |

At the time of use, these compositions are mixed weight for weight with an oxidizing milk assaying 20 volumes of hydrogen peroxide, and then the various mixtures obtained are applied to locks of highly sensitized hair. The leave-in time is set at 30 minutes at ambient temperature. At the end of the leave-in time, the locks are shampooed, rinsed and dried.

Each lock is evaluated before and after dyeing in the L*a*b* system using a CM 2002 Minolta® spectrophotometer (D65 illuminant).

In the L*a*b* system, the three parameters respectively denote the intensity (L*), the hue (a*) and the saturation (b*). According to this system, the higher the value of L, the lighter or less intense the colour. Conversely, the lower the value of L, the darker or more intense the colour. a* and b* indicate two axes of colours: a* indicates the axis of green/red colour and b* the axis of blue/yellow colour.

The calorimetric results given by L* are as follows:

| | L* |
|---|---|
| Locks before colouring | 56.51 |
| Example 1 | 34.1 |
| Example 2 | 19.50 |
| Example 3 | 20.62 |
| Example 4 | 16.80 |
| Example 5 | 16.09 |

These results show the superiority of the colouring with the composition of the present invention.

| | | | CHARACTERIS- |
|---|---|---|---|
| | BLOCK POLYMER | CHARGE | TICS |
| Polymer 1 | PPG-14 Laureth- | Nonionic | Carbamic |
| (comparative) | 60 | | diester of |
| | hexyldicarbamate | | oxypropylenated |
| | (Dapral T sold | | (14 mol) and |
| | by Akzo) | | oxyethylenated |
| | | | (60 mol) lauryl |
| | | | alcohol |
| Polymer 2 | Poly(Styrene/ | Anionic | PS(1500)- |
| (invention) | Acrylic Acid) | | PAA(44 000) |
| Polymer 3 | Poly(Styrene/ | Anionic | PS(4500)- |
| (invention) | Acrylic Acid) | | PAA(22 800) |
| Polymer 4 | Poly(Styrene/β | Cationic | PS(18 600)- |
| (invention) | N-methyl-4- | | P4VPQ(131 300) |
| | Vinyl- | | |
| | Pyridinium | | |
| | iodide) | | |
| Polymer 5 | Poly(β-Ethylene | Cationic | PEO(5900)- |
| (invention) | Oxide/2- | | P2VPQ(56 500) |
| | VinylPyridinium) | | |

1. Dyeing composition comprising, in an appropriate medium,
at least one dye precursor, and
at least one water-soluble or water-dispersible amphiphilic block copolymer comprising at least one hydrophilic block and at least one hydrophobic block without a fatty chain of more than 8 carbon atoms, the hydrophilic block or blocks representing at least 30% by weight of the block copolymer, with the exception of block copolymers of ethylene oxide and of propylene oxide, block copolymers comprising urethane units and block copolymers comprising siloxane units.

2. Composition according to claim 1, in which the hydrophilic block or blocks represent at least 60% by weight of the block copolymer.

3. Composition according to either of the preceding claims, in which the block copolymer is chosen from diblock copolymers, triblock copolymers and multiblock copolymers.

4. Composition according to any one of the preceding claims, in which the block copolymer is linear.

5. Composition according to any one of claims 1 to 4, in which the block copolymer comprises at least two blocks with a hydrophilic nature having different chemical natures, one of them contributing water-solubility or water-dispersibility to the block copolymer.

6. Composition according to any one of claims 1 to 5, in which the block copolymer is chosen from diblock copolymers and triblock copolymers comprising a hydrophilic central block and two hydrophobic side blocks.

7. Composition according to any one of the preceding claims, in which the hydrophilic blocks are formed of water-soluble monomers chosen from anionic water-soluble monomers, nonionic water-soluble monomers and cationic water-soluble monomers, or a mixture of these.

8. Composition according to claim 7, in which the anionic water-soluble monomers are chosen from carboxylic acids comprising ethylenic unsaturation, 2-acrylamido-2-methylpropanesulphonic acid, styrenesulphonic acid, vinylsulphonic acid and vinylphosphonic acid.

9. Composition according to claim 7, in which the nonionic water-soluble monomers are chosen from acrylamide, N—(C₁₋₆ alkylated)- or N,N-di(C₁₋₃ alkylated)acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol, ethylene oxide, hydroxyethyl acrylate, hydroxypropyl acrylate, hydroxyethyl methacrylate and hydroxypropyl methacrylate.

10. Composition according to claim 7, in which the cationic water-soluble monomers are chosen from dimethyldiallylammonium chloride, methylvinylimidazolium chloride, 2-vinylpyridine, 4-vinylpyridine, 2-methyl-5-vinylpyridine, vinylamine, monomers of formula
H₂C═CR₁—CO—X₂
in which
R₁ represents a hydrogen atom or a methyl group,
X₂ represents a linear or branched C₁₋₆ hydrocarbonaceous group carrying at least one primary, secondary or tertiary amine functional group or at least one quaternary nitrogen atom, or a group of formula NHR₂ or of formula NR₂R₃ where R₂ and R₃ each represent, independently of one another, a linear or branched C₁₋₆ hydrocarbonaceous group carrying at least one primary, secondary or tertiary amine functional group or at least one quaternary nitrogen atom.

11. Composition according to any one of the preceding claims, in which the hydrophobic blocks are formed of water-insoluble monomers chosen from vinylaromatic monomers, dienes and alkylated derivatives of dienes, chloroprene, C₁₋₁₀ alkyl, C₆₋₁₀ aryl or C₆₋₁₀ aralkyl acrylates, C₁₋₁₀ alkyl, C₆₋₁₀ aryl or C₁₋₁₀ aralkyl methacrylates, vinyl acetate, vinyl ethers of formula CH₂═CH—O—R and allyl ethers of formula CH₂═CH—CH₂—O—R where R represents a C₁₋₆ alkyl group, acrylonitrile, vinyl chloride, vinylidene chloride, caprolactone, ethylene, propylene, and fluorovinyl monomers or vinyl monomers comprising a perfluorinated chain.

12. Composition according to any one of claims 1 to 10, in which the hydrophilic blocks comprise at most 25 mol % of one or more water-insoluble monomers according to claim 11.

13. Composition according to claim 1 or 12, in which the hydrophobic block or blocks comprise at most 25 mol % of one or more water-soluble monomers according to any one of claims 7 to 10.

14. Composition according to any one of the preceding claims, in which the block polymer or polymers are present in the composition of the present invention in an amount of between 0.01 and 20%, by weight of active material, preferably between 0.1 and 5% by weight of active material, with respect to the weight of the dyeing composition.

15. Composition according to any one of the preceding claims, comprising an oxidation base chosen from para-phenylenediamines, bisphenylalkylenediamines, bis para-aminophenol, para-aminophenols, ortho-aminophenols, heterocyclic bases and their addition salts.

16. Composition according to any one of the preceding claims, comprising a coupler chosen from meta-phenylenediamines, meta-aminophenols, meta-diphenols, naphthalene couplers, heterocyclic couplers, and their addition salts.

17. Composition according to claim 15, in which the amount of each of the oxidation bases is between 0.001 and 10% by weight approximately of the total weight of the dyeing composition.

18. Composition according to claim 16, in which the amount of each of the couplers is between 0.001 and 10% by weight approximately of the total weight of the dyeing composition.

19. Process for the oxidation dyeing of keratinous fibres, comprising the application to the fibres of a dye composition as defined in any one of claims 1 to 18 in the presence of an oxidizing agent for a time sufficient to develop the desired colouring.

20. Process according to claim 19, in which the oxidizing agent is chosen from hydrogen peroxide, urea hydrogen peroxide, alkali metal bromates, persalts, peracids and oxidase enzymes.

21. Process for the oxidation dyeing of keratinous fibres, comprising the application of a dyeing composition comprising a dye precursor as defined in claims 1 to 18, in the presence of an oxidizing agent or of an oxidizing composition, for a time sufficient to develop the desired colouring, this application being followed by the application of the block copolymer as defined in claims 1 to 14.

22. Process according to claim 21, in which the block copolymer is present in the oxidizing composition.

23. Multi-compartment device, in which a first compartment comprises a dyeing composition as defined in any one of claims 1 to 18 and a second compartment comprises an oxidizing agent.

24. Multi-compartment device, in which a first compartment comprises a dyeing composition comprising at least one dye precursor, a second compartment comprising the oxidizing agent and a third compartment which comprises the block copolymer, the dye precursor and the block copolymer being as defined according to any one of claims 1 to 18.

25. Use of the composition defined in claims 1 to 18 for dyeing keratinous fibres.
